(19) United States
(12) Patent Application Publication
(10) Pub. No.: US 2023/0238111 A1
(43) Pub. Date: Jul. 27, 2023

(54) INDIVIDUALIZED MANAGEMENT SYSTEM FOR TREATING GI DISEASES AND METHODS OF USE THEREOF

(71) Applicant: AYBLE HEALTH, INC., Boston, MA (US)

(72) Inventors: Samuel JACTEL, Boston, MA (US); Joseph M. OLSON, Boston, MA (US); Jordan BROWN, Boston, MA (US)

(73) Assignee: AYBLE HEALTH, INC., Boston, MA (US)

(21) Appl. No.: 18/157,749

(22) Filed: Jan. 20, 2023

Related U.S. Application Data
(60) Provisional application No. 63/301,906, filed on Jan. 21, 2022.

(51) Int. Cl. G16H 20/60; G16H 10/60; G16H 10/20; G16H 40/67; G16H 80/00

(52) U.S. Cl. CPC G16H 20/60; G16H 10/60; G16H 10/20; G16H 40/67; G16H 80/00

(57) ABSTRACT

The invention is generally related to Inflammatory Bowel Disease, Irritable Bowel Syndrome and other immune-mediated inflammatory disorders (IMIDs) such as rheumatoid arthritis, the spondyloarthritis disease spectrum, connective tissue disorders, cutaneous inflammatory conditions, asthma and autoimmune neurological diseases such as multiple sclerosis, and more particularly to compositions and methods for management and amelioration of symptoms in a subject in need thereof. One embodiment of the present invention contemplates a system and method useful for managing and ameliorating IBD symptoms resulting in the development of a personalized diet for the IBD participant which will reduce or eliminate most IBD symptoms.

### CROSS-REFERENCE TO RELATED APPLICATIONS

The present invention claims the right of priority of U.S. Provisional Application No. 63/301,906, filed 21 Jan. 2022. The entire disclosures of the above-identified priority applications are hereby fully incorporated herein by reference.

### TECHNICAL FIELD OF THE INVENTION

The invention is generally related to the management of gastrointestinal diseases and other immune-mediated inflammatory disorders (IMIDs) such as Inflammatory Bowel Disease and the like and, more particularly, to systems and methods for identification, management and amelioration of symptoms in a subject in need thereof.

### BACKGROUND OF THE INVENTION

### Inflammatory Bowel Diseases:

The inflammatory bowel diseases [IBD], which include Crohn's disease and ulcerative colitis, are chronic relapsing and remitting diseases that cause inflammation of the gastrointestinal tract.¹ Since the earliest descriptions of ulcerative colitis and Crohn's disease, these inflammatory bowel diseases have grown to be among the most difficult to characterize, difficult to treat, and costly chronic gastrointestinal diseases in the developed world.² Crohn's disease is named after Dr. Burrill B. Crohn, who first described the disease in 1932 along with his colleagues, Dr. Leon Ginzburg and Dr. Gordon D. Oppenheimer. IBD affects an estimated 3 million Americans, with men and women equally likely to be affected by Crohn's disease.³ Although the disease can occur at any age, Crohn's disease is most often diagnosed in adolescents and adults between the ages of 20 and 30, with studies have shown that between 1.5 percent and 28 percent of people with IBD have a first-degree relative, such as a parent, child, or sibling, who also has one of the diseases.⁴ Crohn's disease can affect people from all ethnic backgrounds, although it is more common in Caucasians and recently rates of Crohn's disease have increased among Hispanics and Asians.⁵

Differences amongst the Inflammatory Bowel Diseases can be seen, amongst other factors, based on location of GI tract affected. For example, Crohn's Disease can affect any part of the GI tract from the mouth to the anus, but most commonly affects the end of the small bowel (ileum) and the beginning of the colon. Crohn's affects the entire thickness of the bowel wall, but the resulting inflammation can leave normal areas in between patches of diseased intestine.⁶

The most common type of Crohn's Disease is ileocolitis which affects the terminal ilium, or end of the small intestine, and is characterized by diarrhea, cramping and pain in the middle or lower right part of the abdomen and may be accompanied by significant weight loss. Illeitis is a type of Crohn's that affects only the ileum but exhibits symptoms similar to ileocolitis. Severe cases may include the presence of fistulas or inflammatory abscesses in the right lower quadrant of the abdomen. A third type, known as gastroduodenal Crohn's Disease, affects the stomach and the beginning of the small intestine (duodenum) and is characterized by symptoms including nausea, vomiting, loss of appetite and weight loss. In contrast, jejunoileitis is characterized by patchy areas of inflammation in the upper half of the small intestine (jejunum) and may include mild to intense abdominal pain and cramps following meals and diarrhea and fistulas may form in severe cases or after prolonged periods of inflammation. Crohn's (Granulomatous) Colitis affect only the colon, or large intestine. Common symptoms include diarrhea, rectal bleeding abscess, fistulas and ulcers around the anus. Skin lesions and joint pains are more common in this form of Crohn's than in others.⁷

While the exact etiology of IBD is unknown, the syndrome is characterized by chronic inflammation of the gastrointestinal tract in genetically susceptible individuals exposed to risk factors, including evidence that gut microbiota may play a role.⁸ It is known that the immune system of the gastrointestinal tract plays a major role in providing an appropriate response to harmful pathogens, while inducing an immune tolerance to harmless food materials and commensal flora. That this immune hemostasis is disturbed in patients with IBD is clear and, during the past several decades, epidemiological studies have provided support for the hypothesis that the increase in the incidence of IBD in adults and children may be linked to changes in lifestyle and nutritional habits.⁹ Genetic predisposition plays a key role in vulnerability to Crohn's disease and, to a lesser degree, to ulcerative colitis. These genetic influences account in large part for higher incidences in certain ethnic groups and families.¹⁰ The number of genetic loci associated with different frequencies of IBD is approaching 200, yet in combination they seem to account for less than 15% of variance in Crohn's disease and only 7.5% of variance in ulcerative colitis.¹¹

Additional support indicating the significant role environmental factors play in the development of IBD is illustrated by the disease occurrence pattern evidenced in siblings and in monozygotic twins, the rapidly increasing incidence of IBD over time, and the seismic shifts in incidence among populations of varying ethnicities as they migrate to different geographic areas. Novel technologies have provided an opportunity to scan throughout the genome for finding susceptibility loci associated with IBD. Identification of these variants have elucidated pathways involved in the IBD pathogenesis with the hope for providing better treatments. Recently 163 loci for IBD have been identified, some of which are implicated in other inflammatory diseases; of the 163 loci, 30 loci are specific to CD, 23 loci to UC, and 110 loci are in common.¹²

Most environmental risk factors seem to affect both Crohn's disease and ulcerative colitis in a similar manner, but there are some interesting differences. For example, smoking appears to be protective against ulcerative colitis but worsens the symptoms associated with Crohn's disease, while there is some evidence that appendectomies may be protective against ulcerative colitis but worsen Crohn's disease.¹³

Nor is the pathology of IBD limited to cramping, diarrhea, and the like. It is also recognized that patients with IBD are at an increased risk of developing colorectal cancer, primarily as a result of the intestinal inflammation.¹⁴ Treatment of IBD involves a multidisciplinary approach involving both medical therapies, lifestyle changes and surgical intervention. Recent advances in drug treatments, including the advent of new biologics, has led to advances in the management of IBD. However, these therapies are expensive and, even before the introduction of newer therapeutic agents, treatment cost in the United States from 2003 to 2004 was estimated to exceed $6.3 billion dollars.¹⁵ Even with the introduction of new and expensive treatment modalities, including anti-tumor necrosis factor, Janus kinase (JAK) inhibitors, immune cell modulators, stem cells, lymphocyte sequestration agents, anti-sense drugs, granulocyte cell stimulating factors, and mucosal barrier enhancers, it is not clear that any of these advances will substantially reduce the requirement for surgery to manage IBD.¹⁶

### Irritable Bowel Syndrome:

Similar to IBD, Irritable bowel syndrome (IBS) is a chronic disorder of the gastrointestinal tract, characterized by abdominal pain and alterations in bowel habits¹⁶. Diagnosis is made by a gastroenterologist according to a symptom-based classification system, the Rome Criteria, and patients are categorized according to the predominant bowel habits they experience: IBS-C (constipation), IBS-D (diarrhea), IBS-M (mixed bowel characteristics) or IBS-U (unsubtyped). Quality of life of IBS patients is significantly impacted by symptoms; 40-60% of patients not only experience gastrointestinal distress, but also experience comorbid psychological disorders such as anxiety and depression¹⁷. Other somatic comorbidities include pain syndromes, overactive bladder and migraine.¹⁸

IBS is the most frequently diagnosed chronic gastrointestinal disease. Prevalence rates in North America have been reported to be between 12-15%, but these prevalence numbers may under-report the burden of IBS because of the significant overlap seen in clinical symptoms for this and other GI conditions. For instance, there is a reported 40-50% of IBD patients with concomitant IBS. Pooled global prevalence of IBS is estimated to be over 11% in 2016 and growing, particularly in Asian countries. The most important single risk factors for IBS are female sex, younger age and preceding gastrointestinal infections¹⁶⁻¹⁸.

The financial burden these patients generate on the US healthcare system is significant. Each IBS patient on average generates upwards of $18,000 in direct medical spend each year in the United States and the IBS population contributes significantly to the $140 billion/year in direct spend attributed to gastrointestinal disorders.

As with IBD, the etiology of IBS is unclear and likely multifactorial: gastrointestinal dysmotility, inflammation, visceral hypersensitivity, genetic predisposition, GI infections, and altered intestinal microbiota have all been proposed as mechanisms that contribute to symptomatology. Approaches to treatment are similarly diverse, and can include pharmacologic intervention (e.g., antispasmodics, selective serotonin reuptake inhibitors, opioid agonists, antibiotics, bile salt sequestrants), over-the-counter medication (e.g., peppermint oil, probiotics) and behavioral health management (e.g., dietary alteration, gut-directed psychological therapy). There is no cure and no models exist to predict what combination of therapeutic approaches have the highest likelihood of controlling an individual patients' symptoms¹⁶⁻¹⁸.

Food ingestion is one of the most commonly reported factors that results in the exacerbation of symptoms among patients with IBS and IBD; dietary modifications have the potential to generate significant improvement in patients' quality of life and symptom severity. However, until recently, food-related symptoms had received little attention from the literature, leaving patients to find their own way through the plethora of usually non-validated and untested diagnostic tests and dietary regimens, which could result in clinically relevant nutritional deficits. Although patients with IBS and IBD readily incriminate specific food items as those that are especially likely to precipitate symptoms, only 11-27% of those trigger foods are correctly identified when confirmed in formal, blinded food challenge studies.¹⁸

### Other Immune Mediated Inflammatory Disorders (IMID):

Beyond Inflammatory Bowel Disease and Irritable Bowel Syndrome, a larger classification of conditions labelled immune-mediated inflammatory disorders (IMID) behave similarly and have similar need for novel approaches to management. IMIDs include inflammatory bowel disease as well as other GI diseases like gastroesophageal reflux disease and Barrett's esophagus. IMIDs also include rheumatoid arthritis (RA), osteoarthritis, the spondyloarthritis disease spectrum, connective tissue disorders, cutaneous inflammatory conditions such as psoriasis and atopic dermatitis, asthma, ADHD, Type 1 diabetes, and autoimmune neurological diseases such as multiple sclerosis¹⁹. As a group of conditions, IMIDs have an estimated incidence of 80+ per 100,000 and affect 3-5%+ of the US population.

IMIDs share common underlying pathogenetic features (‘public’ immune pathways), but also present unique (‘private’) pathways that define their clinical phenotype, age and sex distribution, tissue localization and therapeutic response profile, among other characteristics. The genetic overlap between IMIDs can be illustrated by the fact that of the >100 genetic loci associated with RA, only two appear specific to RA and not associated with other IMIDs.

These conditions have no known cures and are often accompanied by various co-morbidities (including cardiovascular disease, metabolic and bone disorders, among others); thus, these conditions pose significant systemic medical challenges as well as financial burden on the healthcare system and US economy^{19,20.} For example, rheumatoid arthritis has been estimated to impact 2-4% of the US population, 20-35% of whom had to stop working within 3 years of disease onset; compared to the general population, individuals with ankylosing spondylitis have lower employment rates, experience more disability, and are absent more frequently from work. As another example, psoriasis patients generate well over $115 M in lost productivity per year as a result of their conditions.²¹

There is a growing body of research on the contribution of diet in the exacerbation of IMID symptoms and the modification of dietary intake as a valid method of IMID management. Given that microbial dysbiosis is a hallmark of many IMIDs and that diet is one of the most important factors shaping the diversity and composition of enteric flora, diet may have the potential to be a preventive and therapeutic strategy²⁰. For example, epidemiological studies suggest that polyunsaturated fatty acids may have a positive effect on rheumatoid arthritis development or the course of multiple sclerosis through their anti-inflammatory and antioxidant properties^{19,22}. As a further example, the Mediterranean Diet, Specific Carbohydrate Diet and even veganism have shown promise for the management of rheumatoid arthritis and psoriasis²¹. Given these results, it's unsurprising that IMIDs are strongly associated with a significant change in normal dietary patters and that the variation is disease-specific²³.

A large majority (60-80%) of persons with Inflammatory Bowel Disease (IBD), irritable bowel syndrome (IBS) or other immune-mediated inflammatory disorders (IMIDs) report that symptoms originate from, or are exacerbated by “trigger foods”, and modify their diets to alleviate discomfort. Although such dietary interventions appear to be a reasonable complementary treatment to traditional clinical care, there is no consensus on what diet is best for IBD, IBS or IMID patients. One major challenge is that these conditions present heterogeneously, and patients often respond differently to the same food, so one-size-fits-all dietary recommendations are not optimal solutions. Additionally, popular diets claiming usefulness as an adjunct to traditional care for IBD and the like, such as the low-fermentable oligosaccharide, disaccharide, monosaccharide, and polyol diet (FODMAP), have notable limitations (e.g., cost, length, complexity) and are not personalized to an individual. Recent advances in smartphone-based digital technologies and machine-learning analysis tools present an opportunity to modernize, simplify, and increasingly personalize the standard approaches to elimination-based dietary intervention for IBD, IBS and IMID patients. Thus, there remains a significant need to develop new approaches for effective management of IBD and other IMIDs.

### SUMMARY OF THE INVENTION

In accordance with the purpose(s) of this invention, as embodied and broadly described herein, one aspect of the present invention relates to systems and methods of use and treatment thereof to manage IMIDs including, but not limited to, IBD and IBS and, especially, to systems and methods that can be used by the patient herself to positively affect outcome by allowing, in one aspect, the identification and confirmation of trigger foods to aid in the personalization of dietary interventions to deliver and maintain potential therapeutic benefits including the amelioration, reduction, or elimination of symptoms.

In one embodiment of the present invention, a personalized care management system is provided which includes methods useful in identifying and confirming triggers associated with exacerbation of symptoms and a system which allows the input and collection of data, the analysis of that data, and a means to communicate outputs and generate personalized treatments which allow an individual to beneficially manage IBD, IBS and/or other IMIDs.

Another embodiment of a personalized care management system provides a system and methods thereof having stages of variable duration, which are contemplated to be iterative, and are useful for a person suffering from an IMID, such as IBD and/or IBS, to positively affect symptom management. In one embodiment, the variable length program is contemplated to last approximately nine (9) weeks.

Yet a further embodiment of the present invention contemplates a digital personalized nutrition tool developed using an iterative methodology by which an IBD, IBS and/or IMID patient can further refine identification and confirmation of specific food triggers to provide an individualized diet eliminating the confirmed trigger foods to aid in positively managing symptoms associated with the IMID condition.

Additional advantages of the invention will be set forth in part in the description which follows, and in part will be obvious from the description, or may be learned by practice of the invention without undue experimentation. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiment(s) of the invention and, together with the description, serve to explain the principles of the invention.

FIG. 1 is an overview of a system in accordance with a method of the present invention whereby individual health information and other inputs are provided, stored and analyzed with the results provided as an output to the participant using the method.

FIG. 2 is a schematic representing a system and method of the present invention and demonstrating the reiterative analysis and processing of information collected from a participant in the present method which provides more accurate outputs to the user.

FIG. 3 is a bar graph representing the percentage of participants in the study of Example 1 who agree with the statements below each bar.

### DETAILED DESCRIPTION OF THE INVENTION

### Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims. Unless defined otherwise, all technical and scientific terms used herein have the same meaning as commonly understood by one of ordinary skill in the art to which this disclosure belongs. Any compositions, methods, and materials similar or equivalent to those described herein can be used in the practice or testing of the present invention. All publications cited herein are incorporated by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Use of the term “about” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. It is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The use of the alternative (e.g., “or”) should be understood to mean either one, both, or any combination thereof of the alternatives.

The term “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “flare” or “flare up” is used generally to refer to a period of acute exacerbated symptoms.

A “subject,” or “individual,” or “patient” as used herein includes any person that can use the program, the system, and the methods contemplated herein to reduce or ameliorate the symptoms associated with any IMID generally, IBD and IBS specifically, or combination thereof.

As used herein “IBD” (Inflammatory Bowel Diseases) and “IBS” (Irritable Bowel Syndrome) are used to generally refer to clinical syndromes involving inflammation of the gastrointestinal tract and include Crohn's Disease including, but not limited to, ileocolitis, illeitis, ileocolitis, jejunoileitis granulomatous colitis, and ulcerative colitis, including ulcerative proctitis, proctosigmoiditis, left-sided colitis and pancolitis, and may be accompanied by a variety of physical signs including, but not limited to, mild to intense abdominal pain, cramps, diarrhea, fistula formation, rectal bleeding, abscess formation, formation of ulcers around the anus, skin lesions and joint pain. Further, it is contemplated that the term IBS includes any IBS subtype, including IBS-C (constipation), IBS-D (diarrhea), IBS-M (mixed bowel pattern) and IBS-U (unsubgrouped).

As used herein “IMID” (immune-mediated inflammatory disorders) includes but is not limited to GI diseases like gastroesophageal reflux disease and Barrett's esophagus, osteoarthritis, rheumatoid arthritis, the spondyloarthritis disease spectrum, connective tissue disorders, cutaneous inflammatory conditions (including psoriasis, eczema and atopic dermatitis), inflammatory bowel disease (IBD), cutaneous inflammatory conditions such as psoriasis and atopic dermatitis, connective tissue disorders, asthma, ADHD (attention deficit hyperactivity disorder), Type 1 diabetes, and autoimmune neurological diseases such as multiple sclerosis.

As used herein “amelioration,” “ameliorate,” “therapy, “treatment” or “treating,” refer to any beneficial or desirable effect associated with a reduction in one or more symptoms or other effects of a disease or condition disclosed herein, such as IBD, IBS or IMIDs in general. For example, in relation to embodiments comprising the treatment of IBD in a subject, “treatment” or “treating” includes any beneficial or desirable effect associated with a reduction in diarrhea, cramping, abdominal pain, frequency of bowel movements, and the like but may also include an increase in subjective indicators such as a feeling of well-being and the presence of more energy. “Treatment” does not necessarily indicate complete eradication or cure of the disease or condition, or associated symptoms thereof.

As used herein, the term “symptom” generally refers to subjective evidence of a disease or disorder, i.e., particularly a feature that is apparent to the individual with the disease or disorder, such as diarrhea or vomiting, while the term “clinical sign” usually indicates objective evidence of a disease or condition, such as fever or the result of a laboratory test. However, as used herein, the terms “symptom” and “clinical sign” may be used interchangeably.

As used herein, “prevent,” and similar words such as “prevented,” “preventing” and the like indicate an approach for preventing, inhibiting, or reducing the likelihood of the occurrence or recurrence of one or more symptoms or other effects of a disease or condition disclosed herein, such as a disease or condition like IBD (including Crohn's Disease and Ulcerative Colitis), IBS or IMIDs. For example, in embodiments that relate to treating IBD in a subject, “prevent,” and similar words such as “prevented,” “preventing” and the like indicate an approach for preventing, inhibiting, or reducing the likelihood of the occurrence of clinical signs and symptoms associated with IBD. Also encompassed within these terms is the delay of the onset or recurrence of a disease or condition or delaying the occurrence or recurrence of the symptoms.

The words “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, “management” or “controlling” one or more symptoms or effects of a disease or condition (e.g., Crohn's Disease) refers to the use of the system or methods contemplated herein to improve the quality of life for an individual by providing better control of symptoms associated with the disease or condition, including diarrhea, cramping, abdominal pain, frequency of bowel movements, and the like.

As used herein, “trigger foods” or “triggers” refer to commonly associated foods that are known, postulated, or identified with the present invention to be associated with an increase in the severity of signs and clinical symptoms associated with IMIDs, IBD (Crohn's Disease and Ulcerative Colitis), IBS and combinations thereof, including but not limited to, rheumatoid arthritis, psoriasis, connective tissue disorders, asthma and multiple sclerosis. Such foods include, but are not limited to, vegetables including legumes like beans, peas, and the like; nightshades, such as tomato, peppers and the like; gluten containing foods including bread, pasta, noodles, cereal, and the like; dairy and lactose-containing foods such as milk and cheese; spicy foods; fried foods; and alcohol. One of ordinary skill in the art will understand that the foods listed above are provided for description and that the disclosure is not limited to the embodiments described but is for the purpose of describing certain embodiments only.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. The description exemplifies illustrative embodiments. In several places throughout the application, guidance is provided through lists of examples, which examples can be used in various combinations. In each instance, the recited list serves only as a representative group and should not be interpreted as an exclusive list. All headings are for the convenience of the reader and should not be used to limit the meaning of the text that follows the heading, unless so specified. It is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in any appropriately detailed structure.

The present invention is directed to methods and systems useful for developing personalized dietary guidance for the identification and elimination of specific trigger foods, resulting in a more positive management of IMIDs such as IBD and IBS. One embodiment of a method according to the present invention generally contemplates an individual participating in four phases after an initial onboarding phase where, amongst other information, demographic data such as age, sex, and ethnicity; medical history including diagnosis, symptoms, and treatment of any condition; medication history; and the like is obtained for the individual. Following the introductory or onboarding phase, the methods of the present invention contemplate four phases in which the individual will participate: an identification phase; an elimination phase; a reintroduction phase; and a maintenance phase. The individual may go through each phase or stage once or may, optionally, go through one or more of the phases in an iterative fashion.

Also contemplated by the present invention is a management system which includes, but is not limited to i) a means for the collection of data, including data such as the occurrence and severity of symptoms self-reported by the individual as the individual participates in the different stages of the method, gathered passively from connected technology such as health wearables (watches, scales, and the like) or from other sources; ii) a means of storing the collected data in a data repository; iii) generation of an individualized database from the data collected and stored at each step or phase of the method of the present invention; iv) analysis of data in the individualized data base using at least one algorithm; v) using the data analysis to develop at least one personalized diet for the individual to follow during at least one stage or phase of the method; and vi) communication of information during one or more stages of the present method including, but not limited to, statistics generated from the collected data; data output; and any change in symptoms or other parameter including subjective indicators measured associated with the condition of concern following institution of at least one personalized diet by the individual. One advantage of the present invention is that, as one of skill in the art can readily appreciate, is that the method is iterative; that is, a participant may repeat at least one step any number of times to achieve the desired result before proceeding through the remainder of the program.

A method in accordance with the present invention useful to ameliorate or reduce symptoms associated with IBD, IBS and/or IMIDs generally consists of four principal phases once onboarding of the participant is completed. One embodiment of a method according to the present invention contemplates including four phases as follows: Identification (Phase 1), Elimination (Phase 2), Reintroduction (Phase 3), and Maintenance (Phase 4) as illustrated in Table 1, as follows:

| | | |
|---|---|---|
| Phase | Week | Activity |

Input: Machine learning algorithm identifies 20-25 foods that may be associated with an individual's
IBD, IBS and/or IMID symptoms to track and validate in phase 1-3.

| | | |
|---|---|---|
| 1 | 1-3 | Patients eat normally and complete daily symptom & diet |
| Identification | | surveys |

Output: Machine learning algorithm down-selects a subset of foods from the initial list of 20-25
foods that may be associated with an individual's IBD, IBS and/or IMID symptoms and suggest they
remove these from their diet in Phase 2. In one embodiment, the subset is 3 to 5 foods.

| | | |
|---|---|---|
| Elimination | 1-2 | Patients remove the suggested foods from their diet entirely |
| | | and complete daily symptom & diet surveys |

Output: Participants establish a baseline to validate that foods identified in Phase 1 are associated
with increased symptom severity as part of Phase 3.

| | | |
|---|---|---|
| 3 | 1-2 | Every 3 days, study participants reintroduce another potential |
| Reintroduction | | trigger food and complete daily surveys throughout, guided by |
| | | a machine learning algorithm that evaluates daily symptoms |

Output: Final description of the least restrictive diet that may measurably improve a participant's
IBD, IBS and/or IMID symptoms

| | | |
|---|---|---|
| 4 | 2 | Patients eat modified diet and complete daily symptom & diet |
| Maintenance | | surveys |

Output: Follow-up monitoring of efficacy of recommended diet

Onboardinq (pre-Phase 1): In this phase, health data is collected from each participant including but not limited to IBD, IBS and/or IMID symptomatology; medication taken; general medical history and the like. In addition, during the Onboarding stage the present invention contemplates the collection of other non-medical data, if desired, including but not limited to demographics such as, but not limited to gender, age, ethnicity; physical activity and the like. Also contemplated is that each participant inputs dietary preferences. Table 2 illustrates additional data that can be collected as follows:

Data Gathered

Third-party (publicly-available or purchased/licensed) data on: Medical
advancements and clinical databases on IBD/IBS/IMID; IBS/IBS/IMID
biomarkers; Dietary intervention data; and the like
Demographic and health information including age; sex; pregnancy
status; race/ethnicity; comorbidities; eating disorders; smoking history;
GI surgeries; Activity of IBD, IBS & IMID symptoms; Modification of
current diet; and the like
Program-related data gathered daily, weekly or after specific phases):
medication adherence; 24 h recall of food items eaten; symptom activity;
self-reported outcome (stress, energy, alertness, and the like)
Technology-related data: User engagement data; Machine learning
optimization data

Analysis includes generating and using machine learning algorithms and providing individual level data analysis; population level data analysis; and additional analysis including clustering by similar patient characteristics such as clinical signs, behavioral manifestations, socioeconomic levels and the like. Statistical analysis is completed according to desired outputs including baseline IBD, IBS and/or IMID symptom scores for each participant, with additional outputs generated that are useful in a method in accordance with the present invention. For example, an embodiment of the invention contemplates calculating user engagement scores. Other outputs of onboarding data collection and analysis include, but are not limited to, initial trigger food recommendations and the generation of at least one hypothesis, to be tracked, validated, and possibly modified in Phase 1 and subsequent phases, as to which foods may be associated with the production and/or worsening of symptoms associated with the individual's condition of concern. Standard references may be consulted to determine the type of analysis methodology to use including, for example, Introduction to Statistical Analysis (1969), Dixon and Massey; McGraw-Hill Publishing, New York and Handbook of Data Analysis (2004), Hardy and Bryman, SAGE Publications (the contents of which are incorporated by reference it their entirety).

Phase 1 (Identification): following introductory Onboarding, a method according to the present invention contemplates that a participant will enter Phase 1 (Identification) which, in one embodiment lasts approximately 3 weeks, during which time each participant eats his or her regular diet. A daily survey will be completed including questions about self-reported GI and/or IMID symptoms (using the symptom scoring methodology as appropriate based on diagnosis) and a 24-hour recall of their unique list of trigger foods eaten. In one preferred embodiment of the present invention, the survey is automated digitally for ease of compliance. A method for a program in accordance with the present invention will also incorporate communication with all participants. In one embodiment of the present invention, communication with participants may be through at least one of a) an encrypted mobile application, b) an encrypted automated texting service, and/or c) secured email or a similar encrypted tool. One embodiment of the Identification Phase (Phase 1) contemplates data inputs including participant health data; non-health participant data; and third party data such as medical test results. Daily food intake data, including tracking results associated with a diet including initial trigger foods and particularly those triggers identified in the initial hypothesis include other examples of data inputs. In one embodiment of the present invention, a participant in preparation for Phase 1 (Identification) will be assigned a unique high-potential set of 20-25 trigger foods to track, based upon personalized factors including diagnosis type, demographic data, symptom severity at intake, and the like; however, it will be recognized by one of ordinary skill that the number of proposed trigger foods may be less or more than the 20-25 foods suggested above, depending upon specific circumstances. Analysis of information collected during Phase 1 or subsequent Phases can be undertaken at various time points. Statistical analysis may include single variable, multivariable, linear or nonlinear regression; clustering by similar patient characteristics; and other methods well known to those skilled in conducting such analyses. For example, one embodiment of the present invention contemplates using a multivariate machine learning algorithm to analyze each participant's individual diet and symptomology to narrow the initial list of potential trigger foods and identify those most strongly associated with adverse symptoms reported during Phase 1. Outputs associated with Phase 1 include, but are not limited to, the refinement of the initial hypothesis as to which foods are implicated as triggers and narrowing the initial list of trigger foods. In one embodiment, the list of triggers is narrowed to 3-5 foods, hypothesized to be most likely responsible for the majority of the symptoms experienced.

Phase 2 (Elimination): In phase 2 which, in one embodiment, lasts approximately two weeks, participants are asked to eliminate the 3-5 trigger foods identified during Phase 1 from their habitual diets. In one embodiment of the Elimination Phase in accordance with the present invention, data inputs during this period of time may include participant health data; non-health participant data; and third party data such as medical test results, similar to data collected during Phase 1. Statistical analysis may be accomplished using a variety of generally accepted methods including multivariable regression; comparative statistics such as a t-test or descriptive statistics. The present invention also contemplates that, in Phase 2, the software used as well as statistical analysis of the data and the participant herself will determine an initial validation of symptom amelioration or improvement following the elimination of certain trigger foods. It should be recognized, however, that a participant could undergo more than one round of the Elimination Phase (Phase 2), if necessary or desirable, to identify which trigger foods, when eliminated, lead to improvement.

Phase 3 (Reintroduction): Following the elimination of certain trigger foods in Phase 2 (Elimination Phase), participants are prompted to reintroduce eliminated foods in the Reintroduction Phase (Phase 3). In one embodiment of the present invention, putative trigger foods are reintroduced one at a time, every three days and wherein each day the participant increases their food intake. It is contemplated that, in one embodiment, this Phase would last about one to two weeks, but it should be recognized that depending on a number of factors including, but not limited to the participant herself, the number of trigger foods eliminated, and the symptoms reported, two weeks is an average time for this Phase to be completed. Just as in Phase 1 and 2, a participant may undergo more than one round of the Reintroduction Phase (Phase 3). Participants complete the same daily digital survey as in Phases 1 and 2. Referring now to FIG. 4, a schematic illustrates one embodiment of the Reintroduction Phase having as data inputs participant health data; non-health participant data; and third party data such as medical test results, amongst other criteria. Statistical analysis is completed using methods similar to those used in earlier Phases. The present invention also contemplates that, as in the earlier Phases, software may be used for statistical analysis of the data collected. Output from this Phase includes a secondary validation of symptom impact; i.e., confirmation of identity of a trigger food if the individual reports or experiences worsening symptoms after reintroduction of that food.

Phase 4 (Maintenance): After the reintroduction phase is complete, one embodiment of the invention contemplates that participants implement the modified diet resulting from removing the trigger foods identified in Phases 1-3 for an additional two weeks. Following Phase 3 (Reintroduction), the present invention contemplates that the participants enter into the final Maintenance stage using implementation and monitoring of the final diet designed by eliminating suspected trigger foods and validating symptom amelioration. Phase 4 is illustrated by FIG. 5 which shows exemplary inputs including participant health data; non-health participant data; and third party data such as medical test results, amongst other criteria, as well as all data collected during Phases 1-3. Statistical methodology also includes descriptive statistics and the like with the principal output from the Maintenance Phase including the generation of a final diet to be implemented in the individual's daily life for the control, amelioration, reduction or elimination of the symptoms and clinical signs associated with the condition of concern. It should be recognized, however, that a participant can and usually will require iterative rounds of Phase 2 to Phase 4 to identify and validate the final diet recommendation.

In one embodiment of the present invention, at study midpoint (end of Phase 2) and study completion, participants also complete qualitative assessments of quality of life and other outcomes. Additional patient-reported outcomes measuring the program's impact on patient understanding of his/her disease and ability to make digestive choices, as well as subjective evaluations of the desirability of the tool, are also assessed and integrated into algorithmic analysis to provide continually improving recommendations aimed at improving the health, wellbeing and quality of life of IBD, IBS and other IMID patients.

In other embodiments, additional phases may be included in a method in accordance with the present invention. For example, in one embodiment a Phase 0 is included which provides a list of 20-25 potential trigger foods identified by conducting a systematic literature review of efficacy studies on diets previously described, clinical practice guidelines, meta-analyses on dietary approaches to IBS and IBD management, and survey studies on the daily dietary practices of patients with IBS and IBD. Data from a total of 4,565 patients with IBS, 1,560 patients with IBD, and 2,993 health controls were included in the analysis. Using these pooled records, an aggregated list of 246 frequently eliminated trigger foods (e.g., alliums, legumes, cultured dairy, and caffeine) was generated and mapped to the aggregated clinical and demographic characteristics (e.g., diagnosis, disease subtype, age, and length of disease course) reported in each study. Guiding principles derived by this relational database were used to supervise a set of sequential weighing, sorting and downsampling algorithms (leveraging feedback-based recursion) to assign a patient a unique set of approximately 21 high-potential trigger foods in Phase 0, which would be tracked by each participant in Phase 1.

In yet a further embodiment of the present invention, the contemplated method may be reiterative. That is, a participant may pass through at least one Phase more than once to achieve desired results.

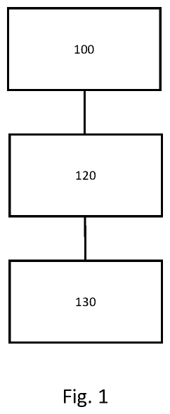

Referring now to the drawings, and initially to FIG. 1, it is pointed out that like reference characters designate like or similar parts throughout the drawings. The Figures, or drawings, are not intended to be to scale. For example, purely for the sake of greater clarity in the drawings, component size and spacing are not dimensioned as they actually exist in the assembled embodiment. FIG. 1, illustrates generally the collection and storage of onboarding data in a data repository and the subsequent analysis of that stored data. In one embodiment of the present invention, FIG. 1 schematically illustrates an input system 100, a processing unit having a data repository and knowledge module 120, and an output system 130 in accordance with one embodiment of the present invention. The input system 100 collects various inputs obtained from a participant including but not limited to personalized health data, information from surveys, data from wearable devices, and the like and the collected information is then transmitted to a processing unit 120 which may include at least one computer processor as well as a knowledge module and a data storage repository. The processing unit comprises computer program instructions for extracting, analyzing, and correlating information relevant to the participant's condition of interest and for the storing of data and personalized results; software programs executable by the processing unit; analyses having methods and algorithms for processing and analyzing data and other inputs including personalized health information; and a program that manages access to resources and services in a digital network. Embodiments of the processing unit can be implemented in one embodiment as software modules installed to run on at least one processing system, such as servers, workstations, tablet computers, PCs, mobile devices and the like. The software applications include a data extraction and analysis application that extracts, identifies and links associated processed data from a knowledge module and data repository and external data sources. An application program interface (API) is also included to permit communication between software programs and the processing unit and for the programs to communicate with each other. The API allows a developer to write a computer program to request services from the operating system (OS) or other application. The interface should be suitable for enabling editing of the knowledge module as, for example, permitting a set of APIs in corresponding libraries to allow the addition, removal or revision of the data within the knowledge module. The processing unit having a knowledge module and data storage repository 120 permits the derivation of further information from existing information by using continual monitoring data as well as crowd-sourced knowledge, inference, and the like. The knowledge module can include one or more databases or data repositories capable of communicating with the knowledge module, but the knowledge module can also receive data from an external data source such as laboratory testing, physician's office, hospital and the like. The data from an external database can be extracted and transferred to the knowledge module using dynamic APIs. The data repository, which stores all information sent to the processing unit, as well as all the data analyzed and correlated by the knowledge module, can be a local storage unit or a remote storage unit. The data repository may be a magnetic storage unit, optical storage unit, solid state storage unit or similar storage unit. The database can be a monolithic device or a distributed set of devices.

The processing unit having a knowledge module and data storage repository 120 processes information accessed and derived by the knowledge module to determine personalized nutritional analytics for participants, including identifying potential trigger foods and providing personalized diet recommendations to confirm the putative foods as triggers as the participant moves through the phases of the Method of the present invention. The processing unit may include one or more algorithms that permit feedback to be provided to the user in real time. For example, the processing unit may include algorithms for predicting trends based upon the client's personalized health profile, diet, and preferences. The analyses may also be stored in a database to be accessed by the knowledge module.

In one embodiment, the processing unit comprises one or more processing devices such as a microprocessor, a central processing unit, and the like. More particularly, the processor may be a complex instruction set computing (CISC) microprocessor, reduced instruction set computing (RISC) microprocessor, very long instruction word (VLIW) microprocessor, or a processor implementing other instruction sets or processors implementing a combination of instruction sets. In another embodiment the processor may be a processing device such as an application specific integrated circuit (ASIC), a field programmable gate array (FPGA), a digital signal processor (DSP), network processor, or the like.

The resulting analyses are transferred to the output system 130 which is in communication with the processing unit having a knowledge module and data storage repository and wherein the output system is configured to periodically advise the participant of needed actions, including dietary changes, as well as to provide reminders, advice and coaching to the participant and/or others such as, but not limited to a medical professional, a health coach, a dietician and the like. It should be appreciated that a system in accordance with the present invention uses and has access to real-time data in order to provide rapid feedback to the participant. Thus, it is contemplated that certain embodiments may be partially implemented using a wireless biometric device that allows data to be automatically uploaded and processed through a processing unit to extract, analyze, and correlate personalized and general information relevant to the participant, such as personalized dietary recommendations to improve the health of the person with the specific condition. The processed data can then be communicated to the participant, and others involved in the participant's care as desired, as instant alerts. As the participant proceeds through the phases of the personalized method of the present invention, additional information can be collected such as, but not limited to, identification and severity of symptoms associated with eating foods recommended by the program activity.

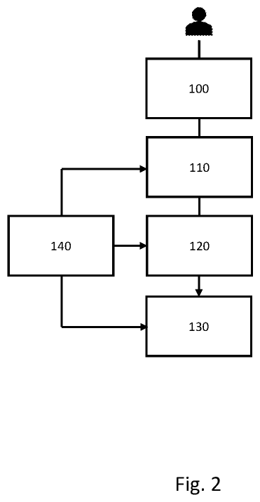

Referring now to FIG. 2, a schematic is provided to illustrate in greater detail an embodiment of a system and method in accordance with the present invention. The system provides the input system 100, the processing unit having a knowledge module and data storage repository 120, and the output system 130 as described above in greater detail. However, also illustrated in FIG. 2 is a portal 110 which serves as the interface between all mechanistic aspects of the invention including but not limited to the processing unit, database(s), and API(s) and the human users of the invention. Portal 110 includes a patient-facing mobile and web application, a professional-facing web application and data visualization toolkit (for the use of physicians, health coaches, dietitians and the like) as well as widgets and other microservices used by machine learning engineers and other software engineering professionals. The mobile and web application embodiments of Portal 110 allow for the direct ingestion of patient-reported and other passively-gathered data, the instantaneous data visualization of this information, the communication of notifications and recommended actions for patients, as well as the interpretation of results generated by the processing unit 120 having the knowledge module and data storage repository during a user's progress through the method and system of the present invention. Portal 110 also allows for participant feedback, including both quantitative and qualitative engagement with the participant, to further customize user experience and health recommendations. Personalization 140 represents analytical techniques used to integrate participant, health coach, physician, dietitian and other qualitative and quantitative feedback on the recommendations provided by using the method and system of the present invention to iteratively improve the health, wellbeing, and quality of life of the IBS, IBD or IMID patient. Also contemplated by an embodiment of this invention is the passive gathering of engagement and user metadata from Portal 110 to further improve recommendations and enhance adherence to those recommendations for users.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art. It is to be understood that the particular examples, materials, amounts, and procedures are to be interpreted broadly in accordance with the scope and spirit of the invention as set forth herein.

### EXAMPLES

### Example 1: Personalized Elimination Diet for Self-Management of Inflammatory Bowel Disease (IBD) and Irritable Bowel Syndrome (IBS)

A decentralized single-center open-labeled uncontrolled prospective cohort study was conducted over nine weeks (63 days). All participants provided written informed consent and received financial compensation and the study and all associated materials were approved by the Advarra Institutional Review Board (protocol #50728). Adults 18-65 years of age previously diagnosed by a gastroenterologist with IBS (meeting Rome III criteria for any subtype of IBS), Crohn's Disease, or Ulcerative Colitis (established by the endoscopic, histologic, and radiologic criteria) or both, with active symptoms, were eligible for inclusion. Minimum symptom activity was determined by the appropriate condition-specific symptom severity score: IBS-SSS≥150 for IBS, sCDAI≥175 and <400 for Crohn's Disease, and p-SCCAI≥2 and <12 for Ulcerative Colitis. Exclusion criteria included pregnancy, significant comorbidities, smoking and disordered eating history, ostomy or known symptomatic intestinal stricture, current use of altered diet, and recent start or change in dose of GI medication.

### Example 2: Intervention

Over a period of nine weeks, participants were guided through four experimental phases of the personalized elimination diet: identification, elimination, reintroduction and maintenance. A link to a secure digital survey was sent to patients each morning of the study period via encrypted text message/SMS and the data was analyzed using machine learning to dynamically guide patients from phase to phase in the following protocol.

Based on a meta-analysis of the literature on the dietary practices of IBD and IBS patients and the diets described previously, the study team generated a database of 246 frequently eliminated “trigger” foods. Based on diagnosis type, demographic data, symptom severity at intake and prior research cited above, each patient was assigned a unique high-potential set of 21 trigger foods to track in phase 1 using a machine-learning algorithm in the onboarding phase.

During phase 1 (3 weeks), participants ate their regular diet and completed a daily digital survey with questions about self-reported GI symptoms (IBS-SSS, p-SCCAI, and mHI-CD as appropriate based on diagnosis) and a 24-hour recall of their unique list of 21 trigger foods eaten. Because there is significant (30-40%) overlap between IBD and IBS diagnosis reported in the literature, IBD patients completed both the IBS-SSS and the appropriate IBD score.

Between phase 1 and phase 2, a multivariate machine learning algorithm was used to analyze each patient's individual diet and symptom data to identify the 3-5 trigger foods most strongly associated with adverse symptoms from the initial list of 21. In phase 2 (2 weeks), participants were asked to eliminate these 3-5 trigger foods from their habitual diets and complete the same daily digital survey, abbreviated to only include this smaller subset of foods. Following phase 2 (elimination), participants were prompted to reintroduce eliminated foods in phase 3, one at a time, every three days, each day increasing food intake by one serving (˜1-2 weeks depending on the individual). Participants completed the same daily digital survey as in phase 1 and 2. If daily symptoms increased in severity participants were prompted to pause the reintroduction for 3 days or until symptoms abated to phase 2 baseline values. After reintroduction, participants implemented the modified diet that they had created for themselves (i.e. removing the trigger foods identified in phase 1-3) for an additional 2 weeks in phase 4.

At study midpoint (end of phase 2) and at completion, participants completed qualitative assessments of their energy/alertness, stress, physical activity, and quality of life. Additional patient-reported outcomes measuring the program's impact on patient understanding of his/her disease and ability to make digestive choices, as well as subjective evaluations of the desirability of the tool were assessed.

### Example 3: Outcome Measures, Statistics and Results

Primary and secondary endpoints were collected at midpoint (week 5) and study completion (week 9). The primary outcome was symptomatic improvement measured in four ways: statistical and clinical significance of symptom improvement, achievement of remission and persistence of symptom amelioration. Symptoms were measured by the appropriate symptom severity score (P-SCCAI for UC, mHI-CD for CD and IBS-SSS for IBS). Statistically significant changes were evaluated by the appropriate statistical analysis. Maintenance of symptom improvement from baseline to end of study were measured to evaluate persistence. Additional self-reported outcomes were also measured, including impact on disease knowledge, overall wellbeing, and quality of life. For these metrics, a majority 60%) of participants responding “Agree” or “Strongly Agree” in a 5-point Likert scale was considered successful.

Secondary endpoints related to this program's feasibility and desirability. Feasibility was measured by participant engagement, retention, and adherence to the program. Engagement was measured by daily completion of surveys and participant retention. Adherence to study protocol was measured by the percentage reduction of trigger food intake and number of days compliant with program recommendations.

Desirability was measured via qualitative satisfaction scores (0-100%) and Net Promoter Score (NPS). The NPS categorizes responders into 3 groups: “promoters” who recommend the tool, “passives” who are happy but but wouldn't actively promote it, and “detractors” who actively discourage others to use it. NPS scores were calculated by subtracting the percentage of detractors from the percentage of promoters (score range −100 to +100). A NPS score 0 and a patient satisfaction score >50% was defined as “desirable”.

Statistical Analysis: To evaluate primary endpoints, repeated measures ANOVAs with post hoc, Bonferroni-corrected, two-tailed paired-samples t-tests were used to compare symptom severity in phase 1 to symptom severity following trigger food elimination and maintenance (phase 2 and 4). Statistical analysis was conducted at both the individual-level and group-level. Descriptive statistics were reported as averages, medians, counts or percentages. Qualitative data were analyzed using McNemar's Chi-square test with Yates' continuity correction. A p value of <0.05 was considered statistically significant for primary and secondary outcomes

To evaluate secondary endpoints, the appropriate descriptive statistics were used; additional qualitative data were analyzed using McNemar's Chi-square test with Yates' continuity correction. All statistical analyses were conducted using web based software known as STATA and JASP.

In one embodiment, statistical analysis was performed prior to program start (pre-phase 1), between phase 1 and phase 2, at the end of phase 2, and daily during phase 3. Prior to study start, a machine learning algorithm determines a set of high-potential trigger foods (“hypothesis”), given the user's unique biological, clinical and socioeconomic characteristics, data gathered from other users and third-party sources. Between phase 1 and 2, a second machine learning algorithm for each participant is used to identify the subset of food categories hypothesized prior to phase 1 to be most likely to cause GI symptoms. At the end of phase 2, a t-test (or other relevant statistical method) can be used to compare symptom severity during phase 1 to symptom severity after trigger food elimination, to verify if symptoms have improved. Finally, a t-test (or other relevant statistical method) can be used to compare symptom severity during the 3 days of trigger food reintroduction in phase 3 to the average symptom severity at the end of phase 2 (i.e. baseline), to verify if symptoms have returned. Additional statistical analysis can be conducted as necessary and desired. At study completion, standard statistical descriptors (such as mean, standard deviation, median and others) can be determined to measure participant engagement. Principal factor analysis can be conducted to identify groupings in patient characteristics/demographics to understand which participants responded best to the protocol used.

Related to data collection, management and statistical analysis, this invention also includes the use and development of computer systems (machine learning algorithms) that are able to learn and adapt without following explicit instructions, by using algorithms and statistical models to analyze and draw inferences from patterns in data. Furthermore, the invention also included a continuous improvement mechanism whereby the data collected and generated from each additional program participant is included in an expanding database. The expanding dataset (including behavioral health, clinical characteristics, user engagement and other metrics) was used to improve the statistical analysis described above using machine learning.

Using the data provided by the participant and collected in the database, a detailed diet specific to each phase and personalized for each participant can be created in accordance with embodiments of the present invention.

The 37-member cohort who completed the study (16 IBS, 12 UC and 9 CD comprised participants that were 62% female, with an average age was 37, and 73% of participants were white. Primary and secondary outcomes showed that the digital personalized elimination diet program improved symptoms statistically and clinically significantly for a majority of participants. Most participants achieved symptomatic remission and symptom relief persisted for the entire study period.

Statistically significant symptom improvement (p<0.001 Bonferroni-corrected two-tailed t-test) was seen for 81% of participants at week 5 and persisted for 70% of participants at week 9, measured by the relevant symptom severity score. By end of study, IBS patients (n=16) improved symptoms by an average 59.3 points (IBS-SSS, p<0.001, petasq=0.62), UC patients (n=12) improved by an average 1.3 points (P-SCCAI, p<0.001, petasq=0.53) and CD patients (n=9) improved by an average 3.1 points (mHI-CD, p<0.001, petasq=0.72), evaluated by one-way repeated-measures ANOVA.

Symptom improvement at midpoint was maintained until the end of the study for patients in each condition group. IBS symptoms at study midpoint (117.6+/−60.4 IBS-SSS) and end (104.9+/−63.0) were significantly lower than baseline (170.6+/−59.3, p<0.001), as were UC symptoms (2.9+/−1.3 P-SCCAI at baseline, 1.9+/−1.3 at midpoint and 1.4+/−1.4 at end, p<0.007) and CD symptoms (6.6+/−1.3 m HI-CD at baseline, 3.0+/−1.9 at midpoint, 3.9+/−1.9 at end, p<0.001), calculated via Bonferroni-corrected post-hoc analysis. No significant difference was found between phase 2 and 4 for any diagnosis group (IBS p=0.66, UC p=0.51, CD p=0.36), indicating symptom relief was maintained successfully 4 weeks after the identification and elimination of personal trigger foods.

Given frequent concomitant IBD and IBS diagnoses, IBS-SSS scores for IBD patients were also evaluated (total n=37) and showed a 49-point improvement from baseline (p<0.001, petasq=0.53, one-way repeated-measures ANOVA). Symptom relief was similarly maintained until study end (154.3+/−52.8 at baseline, 106.9+/−52.9 at midpoint and 103.8+/−54.9 at end, p<0.001). No significant difference was found between Phase 2 and 4 (p=0.99).

Seventy-eight percent of participants saw clinically significant symptom improvement at week 5 and 62% at week 9 (week 5 vs 9 p=0.18, McNemar's test). Twenty-five participants (67.6%) achieved symptomatic remission by end of study (50%, 62.5% and 77.8% of IBS, UC and CD cohorts, respectively). Patients were 14 times more likely to be in remission at study end compared to baseline (p<0.001, McNemar's test). No significant difference was observed between study midpoint and endpoint (p=0.15), indicating a persistence of clinically significant symptomatic relief. Statistically and clinically significant symptom improvement was observed regardless of sex, age quartile, severity at intake and ethnicity.

The results of the 9-week study are summarized in Table 2, as follows:

| | |
|---|---|
| Baseline at intake | End (Phase 4) |

All Participants (IBS-SSS) (n = 37)

| | | |
|---|---|---|
| Severe (>300) | 12 (32.2%) | — |
| Moderate (175 < x < 300) | 17 (45.9%) | 6 (16.2%) |
| Mild (75 < x < 175) | 8 (21.6%) | 15 (40.5%) |
| Remission (<75) | — | 16 (43.2%) |

IBS (IBS-SSS) (n = 16)

| | | |
|---|---|---|
| Severe (>300) | 6 (37.5%) | — |
| Moderate (175 < x < 300) | 8 (50.0%) | 3 (18.7%) |
| Mild (75 < x < 175) | 2 (12.5%) | 5 (31.3%) |
| Remission (<75) | — | 8 (50.0%) |

Ulcerative Colitis (p-SCCAI) (n = 12)

| | | |
|---|---|---|
| Severe (12-19) | — | — |
| Moderate (6-11) | 2 (16.7%) | — |
| Mild (3-5) | 10 (83.3%) | 2 (16.7%) |
| Remission (0-2) | — | 10 (83.3%) |

Crohn's Disease (mHI-CD and s-CDAI) (n = 9)

| | | |
|---|---|---|
| Active Disease (>5.5 mHI- | 9 (100%) | 2 (22.2%) |
| CD, >150 s-CDAI) | | |
| Inactive Disease (>5.5 mHI- | — | 7 (77.8%) |
| CD, <150 s-CDAI) | | |

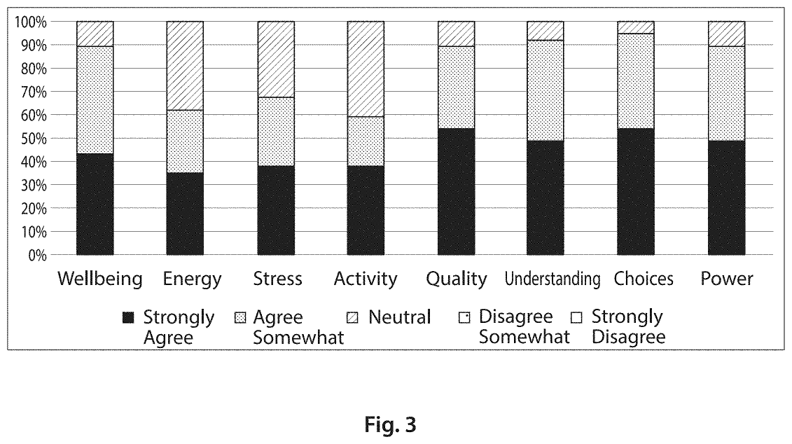

Secondary Outcomes: Patients reported the diet program was feasible and desirable. Ninety-five percent daily engagement (completed surveys) and 89% adherence with the protocol were observed. Participants reduced their intake of suggested trigger foods by 89.3% on average during the elimination phase (89%+/−13%); adherence was significantly left-skewed (skewness=−1.3, SE=0.39). Higher adherence during trigger food elimination was not associated with increased symptom improvement (p=0.62, 0.70, and 0.76 for IBS, UC and CD, respectively) due to homogeneous adherence, see FIG. 4. Eighty-nine percent of participants (n=33) were fully or partially adherent to the reintroduction protocol. Eighty-five percent of those participants (n=28) were able to re-identify at least one trigger food during reintroduction; these individuals reduced intake of trigger foods in the maintenance phase by an average of 65% relative to baseline. Patient-reported outcomes related to disease knowledge, quality of life, overall wellbeing and five other factors also demonstrated strong improvement as is shown in the bar graph in FIG. 3, which details symptom improvement as reported by each participant as a result of the personalized elimination diet program. Each bar illustrated in the graph represents the percentage of participants who agree with the statement below the bar.

A study using the method and system in accordance with the present invention is described fully in Jactel, Samuel N et al; Efficacy of a Digital Personalized Elimination Diet for the Self-Management of Irritable Bowel Syndrome and Co-Morbid Irritable Bowel Syndrome and Inflammatory Bowel Disease. Clinical and Translational Gastroenterology ( ):10.14309/ctg.0000000000000545, Nov. 1, 2022.|DOI: 10.14309/ctg.0000000000000545; ²⁴ the contents of which is incorporated within in its entirety.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention. All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

### REFERENCES CITED

1. Hart, A L et al., What are the top ten research questions in the treatment of inflammatory bowel disease? A priority of setting partnership with the James Linn Alliance. 2017. Journal of Crohn's and Colitis: 204-211.
2. Cohen, B L and D B Sacher, Update on anti-tumour necrosis factor agents and other new drugs for inflammatory bowel disease. 2017. BMJ: 2505.
3. Overview of Crohn's Disease. 2021. The Crohn's and Colitis Foundation, Crohnscolitisfoundation.org/what-is-crohns-disease/overview: 1-5.
4. IBID.
5. IBID
6. IBID
7. Khor B et al. 2011. Genetics and pathogenesis of inflammatory bowel disease. Nature 474: 307-317.
8. Axelrad J E et al. 2016. Inflammatory bowel disease and cancer: the role of inflammation, immunosuppression and cancer treatment. WJG (22): 4794-4801.
9. Hart, A L. Journal of Crohn's and Colitis: 205.
10. Cohen and Sacher, BMJ: 2505.
11. IBID
12. Nemati S and Teimourian S. 2017. An overview of inflammatory bowel disease: general consideration and genetic screening approach in diagnosis of early onset subsets. Middle East J Dig Dis 9: 69-80.
13. Cohen and Sacher, BMJ: 2505
14. Axelrad et al. WJG: 4794.
15. Cohen and Sacher, BMJ: 2505.
16. Weaver K R, Melkus G D, Henderson W A. Irritable Bowel Syndrome. Am J Nurs. 2017; 117(6):48-55. doi:10.1097/01.NAJ.0000520253.57459.01
17. Vahedi H, Ansari R, Mir-Nasseri M, Jafari E. Irritable bowel syndrome: a review article. Middle East J Dig Dis. 2010; 2(2):66-77.
18. Enck, P., Aziz, Q., Barbara, G. et al. Irritable bowel syndrome. Nat Rev Dis Primers 2, 16014 (2016). https://doi.org/10.1038/nrdp.2016.14.
19. McInnes, I. B., Gravallese, E. M. Immune-mediated inflammatory disease therapeutics: past, present and future. Nat Rev Immunol 21, 680-686 (2021). https://doi.org/10.1038/s41577-021-00603-1.
20. Thao Pham, Harry Sokol, Bruno Haliqua et al. Immune-mediated inflammatory diseases and nutrition: results from an online survey on patients' practices and perceptions, 21 Jan. 2021, PREPRINT (Version 1) available at Research Square [https://doi.org/10.21203/rs.3.rs-137887/v1.
21. P. Jacobs et al., Socioeconomic Burden of Immune-Mediated Inflammatory Diseases—Focusing on Work Productivity and Disability. The Journal of Rheumatology Supplement November 2011, 88 55-61; DOI: 10.3899/jrheum.11090.
22. Jiang Y, Jarr K, Layton C, Gardner C D, Ashouri J F, Abreu M T, Sinha S R. Therapeutic Implications of Diet in Inflammatory Bowel Disease and Related Immune-Mediated Inflammatory Diseases. Nutrients. 2021; 13(3):890. https://doi.org/10.3390/nu13030890.
23. Julià, A., Martinez-Mateu, S. H., Domènech, E. et al. Food groups associated with immune-mediated inflammatory diseases: a Mendelian randomization and disease severity study. Eur J Clin Nutr 75, 1368-1382 (2021). https://doi.org/10.1038/s41430-021-00913-6.
24. Jactel, Samuel N et al; Efficacy of a Digital Personalized Elimination Diet for the Self-Management of Irritable Bowel Syndrome and Co-Morbid Irritable Bowel Syndrome and Inflammatory Bowel Disease. Clinical and Translational Gastroenterology ( ):10.14309/ctg.0000000000000545, Nov. 1, 2022.|DOI: 10.14309/ctg. 0000000000000545.

What is claimed is:

1. A system useful for the reduction of clinical symptoms in a person having an immune-mediated inflammatory disease comprising:
a. an input system in communication with a processing unit;
b. a processing unit having a data repository and a knowledge module wherein the knowledge module is in communication with the data repository and has a set of computer program instructions for extracting, analyzing, and correlating information relevant to the immune-mediated inflammatory disease of the person;
c. an output system; and
d. an application program interface (API) in communication with the input system, the processing unit, and the output system, and wherein the API is configured to communicate with the person through an interactive portal.

2. The system according to claim 1, wherein the input system is configured to receive and collect various inputs obtained from a participant including personalized health data, information from surveys, and data from wearable devices and the collected information is then transmitted to a processing unit.

3. The system according to claim 1, wherein the processing unit comprises a knowledge module and a data repository and wherein the knowledge module is configured to receive data from the input system and from an external data source selected from the group comprising laboratory testing, a physician's office, a hospital, a dietician and a coach.

4. The system according to claim 1, further comprising one or more software programs and an API configured to permit communication between the software programs and between a software program and the processing unit.

5. The system according to claim 3, wherein the processing unit further comprises computer program instructions for extracting, analyzing, and correlating data for personalized results relevant to the participant's condition of interest and wherein the data and results are stored in the data repository.

6. The system according to claim 5, wherein the processing unit comprises one or more processing devices such as a microprocessor, a central processing unit, a complex instruction set computing (CISC) microprocessor, a reduced instruction set computing (RISC) microprocessor, a very long instruction word (VLIW) microprocessor, a processor implementing other instruction sets, a processor implementing a combination of instruction sets, an application specific integrated circuit (ASIC), a field programmable gate array (FPGA), a digital signal processor (DSP), and a network processor.

7. The system according to claim 1, wherein the processing unit communicates with an output system configured to periodically advise the participant of needed actions including dietary changes as well as to provide reminders, advice, and coaching to the participant.

8. The system according to claim 1, wherein the immune-mediated inflammatory disease is selected from the group consisting of inflammatory bowel disease, Barrett's esophagus, gastroesophageal reflux disease, rheumatoid arthritis, osteoarthritis, spondyloarthritis, connective tissue disorders, cutaneous inflammatory conditions such as psoriasis and atopic dermatitis, asthma, ADHD, Type 1 diabetes, and autoimmune neurological diseases such as multiple sclerosis.

9. The system according to claim 7, wherein the inflammatory bowel disease is selected from the group consisting of Crohn's disease, including ileocolitis, ileitis, gastroduodenal Crohn's Disease, jejunoileitis, and Crohn's colitis and ulcerative colitis.

10. A method for developing a personalized diet for the reduction of clinical symptoms in a person having an immune-mediated inflammatory disease comprising:
a. collecting personal information from the patient through an input system;
b. transferring the collected information to a processing unit comprising a knowledge module and a data repository and wherein the processing unit is in communication with the input system and the knowledge module is in communication with the data repository;
c. analyzing the collected information in the processing unit using a set of computer program instructions for generating results from extracting, analyzing, and correlating the information relevant to the immune-mediated inflammatory disease of the person;
d. storing the information, data and results in the data repository in communication with an output system;
e. using the results in the data repository to develop at least one personalized diet for the individual to follow;
f. communicating the personalized diet and other information selected from the group comprising data output; statistics generated from the collected data; change in symptoms reported by the participant; and change in subjective indicators associated with the immune-mediated inflammatory disease.

11. The method according to claim 10, wherein the input system is configured to receive and collect various inputs obtained from a participant including personalized health data, information from surveys, and data from wearable devices and the collected information is then transmitted to a processing unit.

12. The method according to claim 10, wherein the processing unit comprises a knowledge module and a data repository and wherein the knowledge module is configured to receive data from the input system and from at least one external data source selected from the group comprising laboratory testing, a physician's office, a hospital, a dietician and a coach.

13. The method according to claim 12, wherein the processing unit further comprises computer program instructions for extracting, analyzing, and correlating data for personalized results relevant to the participant's condition of interest and wherein the data and results are stored in the data repository.

14. The method according to claim 13, wherein the processing unit comprises one or more processing devices such as a microprocessor, a central processing unit, a complex instruction set computing (CISC) microprocessor, a reduced instruction set computing (RISC) microprocessor, a very long instruction word (VLIW) microprocessor, a processor implementing other instruction sets, a processor implementing a combination of instruction sets, an application specific integrated circuit (ASIC), a field programmable gate array (FPGA), a digital signal processor (DSP), and a network processor.

15. The method according to claim 10, wherein the processing unit communicates with an output system configured to periodically advise the participant of needed actions including dietary changes, reminders, advice, and coaching.

16. The method according to claim 10, wherein the immune-mediated inflammatory disease is selected from the group consisting of inflammatory bowel disease, Barrett's esophagus, gastroesophageal reflux disease, rheumatoid arthritis, osteoarthritis, spondyloarthritis, connective tissue disorders, cutaneous inflammatory conditions such as psoriasis and atopic dermatitis, asthma, ADHD, Type 1 diabetes, and autoimmune neurological diseases such as multiple sclerosis.

17. The method according to claim 16, wherein the inflammatory bowel disease is selected from the group consisting of Crohn's disease, including ileocolitis, ileitis, gastroduodenal Crohn's Disease, jejunoileitis, and Crohn's colitis and ulcerative colitis.

18. The method according to claim 10 wherein the personalized diet is developed as the participant participates in at least four phases comprising identification of at least one potential trigger food, elimination of at least one trigger food in a personalized diet; reintroduction of at least one trigger food in the diet to confirm its association with clinical symptoms; and a maintenance stage wherein the confirmed trigger foods are not included in the participant's diet.

19. The method of claim 17, wherein the participant may repeat at least one of the four phases to develop the personalized diet in an iterative manner.

20. The method of claim 18, where at least one additional phase may be included in developing a personalized diet comprising of a pre-onboarding phase by compiling a list of trigger foods identified in literature reviews, survey results, and in clinical practice guidelines.
